(19) United States
(12) Patent Application Publication
(10) Pub. No.: US 2010/0249552 A1
(43) Pub. Date: Sep. 30, 2010

(54) System And Method For Wirelessly Powering Medical Devices

(75) Inventor: Thomas Price, Edgewater, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/414,974

(22) Filed: Mar. 31, 2009

(51) Int. Cl. A61B 5/1455; H01F 27/42

(52) U.S. Cl. 600/324; 307/104

(57) ABSTRACT

A system and method for the wirelessly charging of a power source of a pulse oximeter. The pulse oximeter may include an inductively coupled conductor. The inductively coupled conductor may be coupled to the power source and the inductively coupled conductor may wirelessly receive an electromagnetic charging signal. Based on the received signal, the inductively coupled conductor may at least partially recharge the power source.

### BACKGROUND

The present disclosure relates generally to medical devices and, more particularly, to powering those devices wirelessly.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable pail of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed and/or scattered. More specifically, the light passed through the tissue is typically selected to be of one or more wavelengths that may be absorbed and/or scattered by the blood in an amount correlative to the amount of the blood constituent present in the blood. The amount of light absorbed and/or scattered may then be used to estimate the amount of blood constituent in the tissue using various algorithms.

Traditional pulse oximeters obtain power by plugging into a wall socket. However, a wall socket may not be conveniently located near a patient for use in obtaining power. The use of batteries in a pulse oximeter may address this problem, however the batteries in such pulse oximeters require regular recharging or replacement. In situations where recharging facilities or replacement batteries are not readily available, these pulse oximeters become similarly disadvantaged as the traditional plug-in pulse oximeters.

### BRIEF DESCRIPTION OF THE DRAWINGS

Advantages of the disclosure may become apparent upon reading the following detailed description and upon reference to the drawings in which:

FIG. 1 illustrates a perspective view of a pulse oximeter in accordance with an embodiment;

FIG. 2 illustrates a simplified block diagram of a pulse oximeter in FIG. 1, according to an embodiment;

FIG. 3 illustrates a wireless inductive power system including the pulse oximeter of FIG. 1, according to an embodiment; and

FIG. 4 illustrates a block diagram of the inductive power system of FIG. 3.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A system and method for wirelessly powering a pulse oximeter is provided herein. The system may include a charging station, which may generate electromagnetic charging signals. The pulse oximeter may include an inductive coil that may receive the generated electromagnetic charging signals, and may utilize the electromagnetic charging signals to generate electricity via an inductor in the pulse oximeter. This electricity may be utilized for the operation of the pulse oximeter, or, alternatively, for the charging of a power source, such as a rechargeable battery, in the pulse oximeter. Additionally, the charging station and the pulse oximeter may include control circuitry that may transmit various signals to the charging station that activate and deactivate the charging station based on the charging requirements of the oximeter.

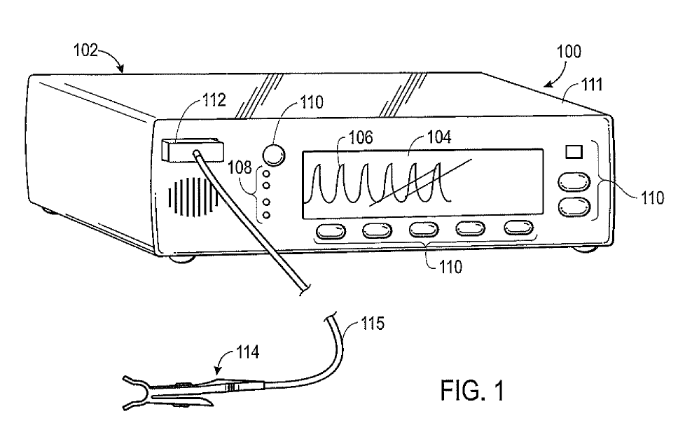

Turning to FIG. 1, a perspective view of a medical device is illustrated in accordance with an embodiment. The medical device may be a pulse oximeter 100. The pulse oximeter 100 may include a monitor 102, such as those available from Nellcor Puritan Bennett LLC. The monitor 102 may display calculated parameters on a display 104. As illustrated in FIG. 1, the display 104 may be integrated into the monitor 102. However, the monitor 102 may provide data via a port to a display (not shown) that is not integrated with the monitor 102. The display 104 may display computed physiological data including, for example, an oxygen saturation percentage, a pulse rate, and/or a plethysmographic waveform 106. As is known in the art, the oxygen saturation percentage may be a functional arterial hemoglobin oxygen saturation measurement in units of percentage Sp_{O2}, while the pulse rate may indicate a patient's pulse rate in beats per minute. The monitor 102 may also display information related to alarms, monitor settings, and/or signal quality via indicator lights 108.

To facilitate user input, the monitor 102 may include a plurality of control inputs 110. The control inputs 110 may include fixed function keys, programmable function keys, and soft keys. Specifically, the control inputs 110 may correspond to soft key icons in the display 104. Pressing control inputs 110 associated with, or adjacent to, an icon in the display may select a corresponding option. The monitor 102 may also include a casing 111. The casing 111 may aid in the protection of the internal elements of the monitor 102 from damage.

The monitor 102 may further include a sensor port 112. The sensor port 112 may allow for connection to an external sensor 114, via a cable 115 which connects to the sensor port 112. The sensor 114 may be of a disposable or a non-disposable type. Furthermore, the sensor 114 may obtain readings from a patient, which can be used by the monitor to calculate certain physiological characteristics such as the blood-oxygen saturation of hemoglobin in arterial blood, the volume of individual blood pulsations supplying the tissue, and/or the rate of blood pulsations corresponding to each heartbeat of a patient.

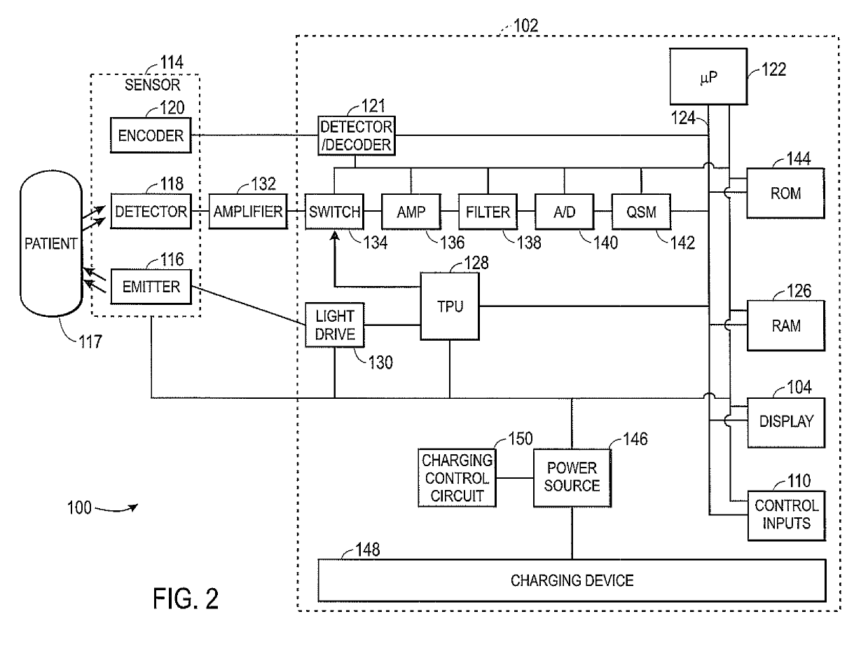

Turning to FIG. 2, a simplified block diagram of a pulse oximeter 100 is illustrated in accordance with an embodiment. Specifically, certain components of the sensor 114 and the monitor 102 are illustrated in FIG. 2. The sensor 114 may include an emitter 116, a detector 118, and an encoder 120. It should be noted that the emitter 116 may be capable of emitting at least two wavelengths of light, e.g., RED and infrared (IR) light, into the tissue of a patient 117 to calculate the patient's 117 physiological characteristics, where the RED wavelength may be between about 600 nanometers (nm) and about 700 nm, and the IR wavelength may be between about 800 nm and about 1000 nm. Alternative light sources may be used in other embodiments. For example, a single wide-spectrum light source may be used, and the detector 118 may be capable of detecting certain wavelengths of light. In another example, the detector 118 may detect a wide spectrum of wavelengths of light, and the monitor 102 may process only those wavelengths which are of interest for use in measuring, for example, water fractions, hematocrit, or other physiologic parameters of the patient 117. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

Additionally the sensor 114 may include an encoder 120, which may contain information about the sensor 114, such as what type of sensor it is (e.g., whether the sensor is intended for placement on a forehead or digit) and the wavelengths of light emitted by the emitter 116. This information may allow the monitor 102 to select appropriate algorithms and/or calibration coefficients for calculating the patient's physiological characteristics. The encoder 120 may, for instance, be a memory on which one or more of the following information may be stored for communication to the monitor 102; the type of the sensor 114; the wavelengths of light emitted by the emitter 116; and the proper calibration coefficients and/or algorithms to be used for calculating the patient's 117 physiological characteristics. The sensor 114 may be any suitable physiological sensor, such as those available from Nellcor Puritan Bennett LLC.

Signals from the detector 118 and the encoder 120 (if utilized) may be transmitted to the monitor 102. The monitor 102 may include one or more processors 122 coupled to an internal bus 124. Also connected to the bus may be a RAM memory 126 and a display 104. A time processing unit (TPU) 128 may provide timing control signals to light drive circuitry 130, which controls when the emitter 116 is activated, and if multiple light sources are used, the multiplexed timing for the different light sources. TPU 128 may also control the gating-in of signals from detector 118 through an amplifier 132 and a switching circuit 134. These signals are sampled at the proper time, depending at least in part upon which of multiple light sources is activated, if multiple light sources are used. The received signal from the detector 118 may be passed through an amplifier 136, a low pass filter 138, and an analog-to-digital converter 140 for amplifying, filtering, and digitizing the electrical signals the from the sensor 114. The digital data may then be stored in a queued serial module (QSM) 142, for later downloading to RAM 126 as QSM 142 fills up. In an embodiment, there may be multiple parallel paths of separate amplifier, filter, and A/D converters for multiple light wavelengths or spectra received.

In an embodiment, based at least in part upon the received signals corresponding to the light received by detector 118, processor 122 may calculate the oxygen saturation using various algorithms. These algorithms may use coefficients, which may be empirically determined, and may correspond to the wavelengths of light used. The algorithms may be stored in a ROM 144 and accessed and operated according to processor 122 instructions.

The monitor 102 may also include a power source 146 that may be used to transmit power to the components located in the monitor 102 and/or the sensor 114. In one embodiment, the power source 146 may be one or more batteries, such as a rechargeable battery. The battery may be user-removable or may be secured within the housing of the monitor 102. Use of a battery may allow the oximeter 100 to be highly portable, thus allowing a user to carry and use the oximeter 100 in a variety of situations and locations. Additionally, the power source 146 may include AC power, such as provided by an electrical outlet, and the power source 146 may be connected to the AC power via a power adapter through a power cord (not shown). This power adapter may also be used to directly recharge one or more batteries of the power source 146 and/or to power the pulse oximeter 100. In this manner, the power adapter may operate as a charging device 148.

In another embodiment, the charging device 148 may alternately and/or additionally include a wireless charging apparatus. For example, the charging device 148 may include an inductor that wirelessly receives electromagnetic charging signals and generates electrical current as a result of the received electromagnetic charging signals. That is, a current may be electrically induced in the charging device 148 wirelessly. This current may optionally be utilized to directly recharge one or more batteries of the power source 146 and/or to power the pulse oximeter 100. Accordingly, the charging device 148 may allow for the pulse oximeter to be used in situations where a power outlet is unavailable near a patient 117.

As may be seen in FIG. 2, the charging device 148 may be positioned lengthwise across the monitor 102, so as to maximize the length of the charging device 148 to aid in increasing the distance at which the charging device 148 may receive and utilize electromagnetic charging signals. In one embodiment, the charging device 148 may be approximately 9 to 10 inches in length. Furthermore, the charging device 148 may be integrated into monitor 102, or, alternatively, the charging device 148 may be affixed externally to the enclosure 111 of the pulse oximeter 100.

The monitor 102 may also include a charging control circuit 150, which may, for example, allow for the adaptive control of an external charging station. The charging control circuit 150 may, for example, include a processing circuit and a transmitter. In one embodiment, the processing circuit may include the processor 122. In another embodiment, the processing circuit may be a separate processor from the processor 122. Regardless, the processing circuit may determine the current level of charge remaining in the power source 146, and may transmit a request, via the transmitter in the charging control circuit 150, for a charging station external to the oximeter 100 to transmit the wireless electromagnetic charging signals used by the charging device 148 to generate an electrical current.

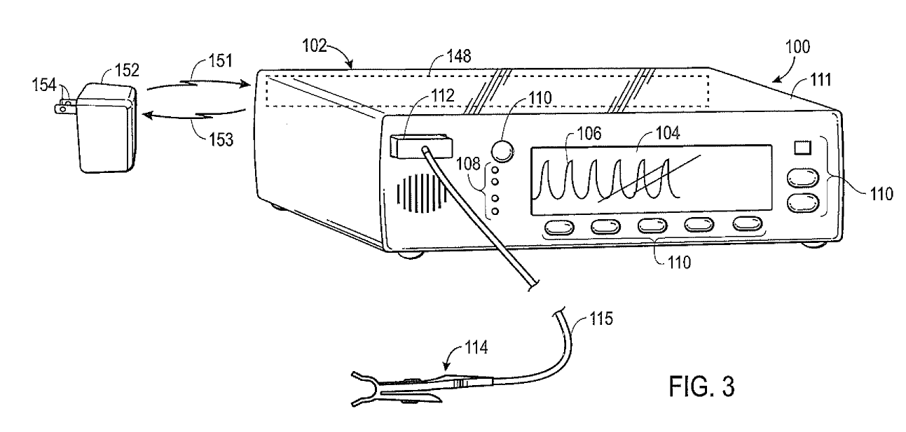

The charging control circuit 150 may also, for example, determine if the charging device 148 is unable to charge the power source 146, for example, if a charging station is failing to generate electromagnetic charging signals for charging of the power source 146, and may generate a corresponding error message for display on the monitor 102. The error message may indicate to a user that the pulse oximeter 100 is low on power and may also direct the user to plug the pulse oximeter 100 into an outlet via the power adapter. This error message may be generated when the charging control circuit 150 determines that the power source 146 has reached a certain charge level, for example, 20% of the total charge remains in the power source 146. The charging control circuit 150 may also perform a handshake recognition function with a charging station, as described below with respect to FIG. 3.

A pulse oximeter 100 that may receive electromagnetic charging signals 151 from a charging station 152, as well as communicate wirelessly 153 with the charging station 152 is illustrated in FIG. 3. The wireless communication 153 that may take place between the pulse oximeter 100 and the charging station 152 may include a handshake recognition function whereby the control circuit 150 of the pulse oximeter 100 may transmit an identification signal to the charging station 152. This identification signal may, for example, be a radio-frequency identification (RFID) that identifies the pulse oximeter 100 as a device for use with the charging station 152. Until this identification signal is received, the charging station 152 may remain in an “off” state, i.e., not transmitting wireless electromagnetic charging signals 151. The charging station 152 may remain “off”, for example, to reduce overall power consumption until a compatible device is within the range of transmission. Thus, the handshake recognition function between the pulse oximeter 100 and the charging station 152 may operate to activate and deactivate the charging station 152.

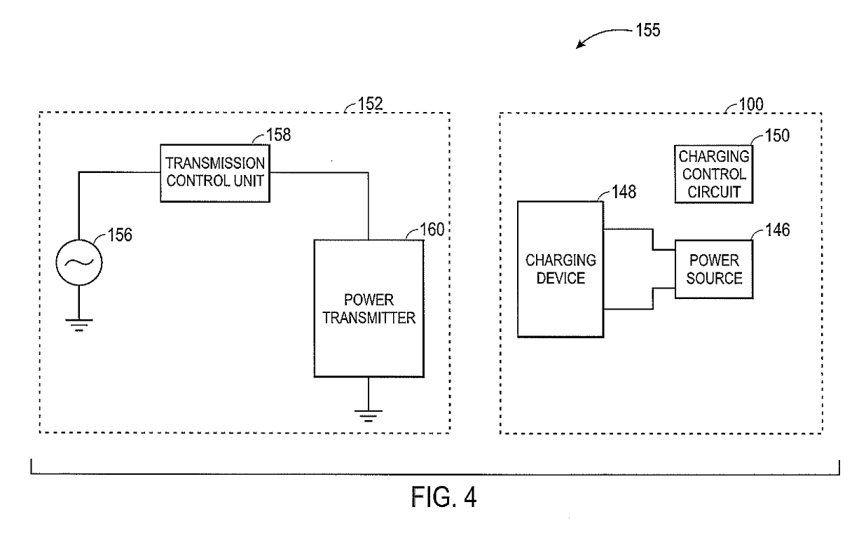

Once a proper identification signal is received, the charging station 152 may be placed into the “on” state. In the “on” state, the charging station 152 may generate and broadcast electromagnetic charging signals 151 based on power received via prongs 154 from a power outlet. These prongs 154 may be affixed to the body of the charging station 152 or, alternatively, the prongs 154 may be connected to the charging station 152 via a power cord. Regardless, the prongs 154 may act to receive power from a power outlet for eventual generation of electromagnetic charging signals 151 by the charging station 152 when requested by the pulse oximeter 100, as described below with respect to FIG. 4.

The block diagram of FIG. 4 illustrates the components of the charging station 152 and the pulse oximeter 100 that may combine to form a wireless inductive power system 155. As illustrated, the pulse oximeter 100 may include a power source 146, a charging device 148, and a charging control circuit 150. The charging station 152 may include an alternating current (AC) power converter 156, a transmission control unit 158, and a power transmitter 160. The AC power converter 156 may represent the power that is received from a wall outlet via prongs 154. This power may be ultimately be transmitted to the power transmitter 160 via the transmission control unit 158.

The transmission control unit 158 may include a receiver and a processing unit. The receiver may receive an identification signal from the charging control circuit 150, and may, as described above, enter an “on” state. Once in the “on” state, the processing unit of the transmission control unit 158, which may be a processor, may await a power transmission request from the charging control circuit 150 of the pulse oximeter. The charging control circuit 150 may, for example, monitor the charge level of the power source 146 and may transmit a power transmission request when the stored charge of the power source 146 reaches a certain threshold, for example, 40% of the total charge of the power source 146.

Once both the identification signal and the power transmission request, i.e., the wireless communications 153, have been received by the transmission control unit 158, the transmission control unit may allow power to flow to the power transmitter 160. The transmission control unit 158 may continue to allow power to flow to the power transmitter until a halt power transmission signal is received from the charging control circuit 150. The halt power transmission signal may be generated and transmitted by the charging control circuit 150 when, for example a threshold of charge level is met in the power source 146. For example, this threshold may be approximately 95% of a full charge of the power source 146. Once a halt signal is received, the charging control circuit 150 may operate to prevent the flow of power to the power transmitter 160, thus ending the wireless power transmission to the pulse oximeter 100 until a power transmission request is received again. In this manner, the pulse oximeter 100 may control the charging of the power source 146 wirelessly. Various wireless powering techniques will be described below.

The power transmitter 160 and the charging device 148 may together form a transformer, that is, an energy transfer mechanism whereby electrical energy is transmitted from the power transmitter 160 to the charging device 148 through inductively coupled conductors. In one embodiment, the inductively coupled conductors may be solenoids, i.e., a metal coil, in each of the power transmitter 160 and the charging device 148. Specifically, a change in current in the inductively coupled conductor of the power transmitter 160 induces a voltage in the conductor of the charging device 148 via generated electromagnetic charging signals 151. However, because the charging signals may radiate in all directions, the intensity may drop off rapidly. Accordingly, the pulse oximeter 100 may only be able to be charged when it is at a distance of approximately the length of the charging device 148, i.e. within a distance approximately equal to the length of the inductively coupled conductor of the charging device 148. To increase this distance, resonant inductive coupling techniques may be utilized.

Resonant inductive coupling may aid in increasing the transmission distance of the electromagnetic charging signals 151 through the use of at least one capacitor in conjunction with the inductively coupled conductor of the power transmitter 160 and/or the charging device 148. For example, a capacitor and the inductively coupled conductor of the power transmitter may form an LC circuit that may be “tuned” to transmit electromagnetic charging signals 151 at a frequency that resonates with the natural resonance frequency of the inductively coupled conductor of the charging device 148. That is, as electricity travels through the inductively coupled conductor of the charging device 148, the conductor resonates as a product of the inductance of the conductor and the capacitance of the one or more capacitors.

In this manner, energy may be generated at a specified “tuned” frequency that allows for focused energy generation at a specific frequency. By generating energy at this specific frequency, instead of at a plurality of frequencies, the generated electromagnetic charging signal 151 will be stronger, thus allowing for increased range of transmission, For example, by utilizing resonant inductive coupling techniques, the transmission range of the electromagnetic charging signals 151 may increase to approximately 3 to 4 times the length of the inductively coupled conductor of the charging device 146. This distance may allow for a single charging station 152 to be placed, for example, in a wall between two rooms in a hospital or clinic, such that a single charging station 152 might provide wireless power to oximeters 100 in each room. This range would also allow for greater ease in placement of an oximeter 100 near a patient 117 regardless of whether there is a power outlet near the patient 117.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims.

What is claimed is:

1. A pulse oximeter comprising:
a power source adapted to power the pulse oximeter; and
an inductively coupled conductor adapted to receive a wireless electromagnetic charging signal and charge the power source at least in part via the wireless electromagnetic charging signal.

2. The pulse oximeter, as set forth in claim 1, comprising a control circuit capable of transmitting an identification signal to initialize the transmission of the wireless electromagnetic charging signal.

3. The pulse oximeter, as set forth in claim 2, wherein the control circuit is capable of monitoring an amount of charge for the power source and transmitting a power transmission request signal for generating the wireless electromagnetic charging signal when the power source reaches a threshold charge level.

4. The pulse oximeter, as set forth in claim 2, wherein the control circuit is capable of monitoring an amount of charge for the power source and transmitting a halt power transmission signal for stopping the generation of the wireless electromagnetic charging signal when the power source reaches a threshold charge level.

5. The pulse oximeter, as set forth in claim 2, wherein the control circuit is capable of generating an error indication message if the power source reaches a threshold charge level.

6. The pulse oximeter, as set forth in claim 5, comprising a display capable of displaying the error indication message.

7. The pulse oximeter, as set forth in claim 1, wherein the inductively coupled conductor comprises a solenoid.

8. The pulse oximeter, as set forth in claim 1, comprising a resonant inductive charging device comprising the inductively coupled conductor and at least one capacitor coupled to the inductively coupled conductor.

9. A wireless inductive power system, comprising:
a pulse oximeter comprising:
a power source adapted to power the pulse oximeter; and
a charging device capable of receiving an electromagnetic charging signal and charging the power source at least in part via the electromagnetic charging signal; and
a charging station capable of generating and wirelessly transmitting the electromagnetic charging signal to the charging device.

10. The wireless inductive power system of claim 9, wherein the charging device comprises a resonant inductive charging device comprising a solenoid and at least one capacitor coupled to the solenoid.

11. The wireless inductive power system of claim 9, wherein the charging station comprises a power transmitter comprising a resonant inductive charging device comprising a solenoid and at least one capacitor coupled to the solenoid.

12. The wireless inductive power system of claim 9, wherein the pulse oximeter comprises a control circuit capable of:
transmitting an identification signal to initialize the transmission of the wireless electromagnetic charging signal;
monitoring an amount of charge for the power source and transmitting a power transmission request signal for generating the wireless electromagnetic charging signal when the power source reaches a first threshold charge level; and
monitoring the amount of charge for the power source and transmitting a halt power transmission signal for stopping the generation of the wireless electromagnetic charging signal when the power source reaches a second threshold charge level.

13. The wireless inductive power system of claim 12, wherein the charging station comprises a receiver capable of receiving the identification signal, the power transmission request, and the halt power transmission signal.

14. The wireless inductive power system of claim 13, wherein the charging station comprises a processor capable of activating and deactivating the transmission of the electromagnetic charging signal based at least in part on each of the identification signal, the power transmission request, and/or the halt power transmission signal, and/or combinations thereof.

15. The wireless inductive power system of claim 9, wherein the pulse oximeter comprises:
a sensor comprising:
a light emitting diode capable of transmitting electromagnetic radiation; and
a photodetector capable of detecting the electromagnetic radiation and generating electrical signals based at least in part upon the detected electromagnetic radiation; and
a monitor coupled to the sensor, wherein the monitor is configured to measure physiological parameters of a patient based at least in part on the electronic signals generated by the sensor.

16. A method comprising:
receiving a wireless electromagnetic charging signal in a pulse oximeter; and
charging a power source of the pulse oximeter at least in part via the wireless electromagnetic charging signal.

17. The method of claim 16, comprising tuning the wireless electromagnetic charging signal at the pulse oximeter based at least in part upon the natural resonance frequency of an inductively coupled conductor of the pulse oximeter.

18. The method of claim 16, comprising transmitting the wireless electromagnetic charging signal at least in part via a charging station external to the pulse oximeter.

19. The method of claim 18, comprising:
transmitting an identification signal from the pulse oximeter to initialize the transmission of the wireless electromagnetic charging signal;
monitoring an amount of charge for the power source at the pulse oximeter and transmitting a power transmission request signal from the pulse oximeter for generating the wireless electromagnetic charging signal when the power source reaches a first threshold charge level; and
monitoring the amount of charge for the power source at the pulse oximeter and transmitting a halt power transmission signal from the pulse oximeter for stopping the generation of the wireless electromagnetic charging signal when the power source reaches a second threshold charge level.

20. The method of claim 19, comprising receiving the identification signal, the power transmission request, and/or the halt power transmission signal at a charging station and modifying the transmission of the electromagnetic charging signal based at least in part on the identification signal, the power transmission request, and/or the halt power transmission signal at the charging station, and/or combinations thereof.
